(19) United States
(12) Patent Application Publication
(10) Pub. No.: US 2015/0182755 A1
(43) Pub. Date: Jul. 2, 2015

(54) Biological Tissue Stimulation of the Auto Immune System Cellular Reaction by Using Optical Energy

(71) Applicant: Gary John Bellinger, Dallas County, TX (US)

(72) Inventor: Gary John Bellinger, Dallas County, TX (US)

(21) Appl. No.: 14/145,356

(22) Filed: Dec. 31, 2013

(51) Int. Cl. A61N 5/06

(52) U.S. Cl. CPC A61N 5/0613; A61N 2005/067

(57) ABSTRACT

The present invention utilizes a hand piece featuring a laser device which is used in the irradiation of damaged cells to stimulate photoreceptors within a cell, initiating a cascade of intracellular metabolic reactions that move the cell toward homeostasis. As the cell approaches homeostasis there occur a number of secondary effects of photo stimulation which contribute to homeostatic cellular metabolism of adjacent cells. Concurrent photo stimulation of the maximal three dimensional volume of cells along with the secondary effects of the cellular metabolic homeostasis achieved in these cells results in a resolution of metabolic deficits within cells far in excess of the effects of direct photo stimulation. The current invention offers an effective method of irradiation of large numbers of cells with safe levels of optical energy to initiate the intracellular cascade toward homeostasis and the production of secondary effects contributing to homeostasis of adjacent or distant cells.

### CROSS-REFERENCE TO RELATED APPLICATIONS

Provisional Patent Application 61/747,745 filed Dec. 31, 2012

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The present invention relates generally to the treatment of living biological tissue by optical irradiation. Furthermore, it is a method for stimulating soft, living tissue by laser irradiation.

2. Description of Related Art

Various non-surgical means have been employed in the therapeutic treatment of living tissue. Such techniques have included the application of ultrasonic energy, electrical stimulation, high frequency stimulation by diathermy, X-rays and microwave irradiation. Techniques such as electrical stimulation, diathermy, X-ray and microwave radiation have shown some therapeutic benefit for soft tissues. However, their use has been somewhat limited because of tissue damage caused by excessive thermal effects. Consequently, the energy levels associated with therapeutic treatments involving diathermy, X-ray, microwave and electrical stimulation have been limited to such low levels that little or no benefit has been obtained. Moreover, the dosage of exposure to microwaves and X-ray radiation must be carefully controlled to avoid radiation related health problems. Ultrasonic energy is non-preferentially absorbed and affects all of the surrounding tissue.

Optical energy generated by lasers has been applied for various medical and surgical purposes because of the monochromatic and coherent nature of laser light which can be selectively absorbed by living tissue depending upon certain characteristics of the wavelength of the light and properties of the irradiated tissue, including reflectivity, absorption coefficient, scattering coefficient, thermal conductivity and thermal diffusion constant. The reflectivity, absorption coefficient and scattering coefficient are dependent upon the wavelength of the optical radiation. The absorption coefficient is known to depend upon such factors as interband transition, free electron absorption, grid absorption (phonon absorption), and impurity absorption, which are dependent upon the wavelength of the optical radiation.

In living tissue, water is a predominant component which has an absorption band according to the vibration of water molecules in the infrared range. In the visible range, there exists absorption due to the presence of hemoglobin. Further, the scattering coefficient in living tissue is a dominant factor.

Thus, for a given tissue type, the laser light may propagate through the tissue, substantially unattenuated, or may be almost entirely absorbed. The extent to which the tissue is heated and ultimately destroyed depends on the extent to which it absorbs the optical energy. It is generally preferred that the laser light be essentially transmissive in tissues which are desired not to be affected, and absorbed by the tissues which are to be affected. For example, when applying laser radiation in a tissue field which is wet with blood or water, it is desired that the topical energy not be absorbed by the water or blood, thereby permitting the laser energy to be directed specifically to the tissue to be treated. Another advantage of laser treatment is that the optical energy can be delivered to the treatment tissues in a precise, well defined location and at predetermined, limited energy levels.

### BRIEF SUMMARY OF THE INVENTION

Therefore it is the object of the present invention to provide a method of generally percutaneously stimulating, in a non-discriminatory fashion intracellular, energy homeostasis. The present invention utilizes a hand piece featuring a laser device which is used in the irradiation of damaged cells. Through the stimulation of photoreceptors within a cell, a cascade of intracellular metabolic reactions are initiated that move the cell toward homeostasis. As a cell approaches homeostasis there occur a number of secondary effects of photo stimulation which contribute to homeostatic cellular metabolism of adjacent cells. Concurrent photo stimulation of the maximal three dimensional volume of cells along with the secondary effects of the cellular metabolic homeostasis achieved in these cells results in a resolution of metabolic deficits within cells far in excess of the effects of direct photo stimulation. The current invention offers an effective method of irradiation of large numbers of cells with safe levels of optical energy to initiate the intracellular cascade toward homeostasis and the production of secondary effects contributing to homeostasis of adjacent or distant cells. This characteristic exponential benefit of photo stimulation is especially valuable in the treatment and stimulation of the blood and immune cells of the body.

These and other objects, advantages and features of this invention will be apparent from the following description taken with reference to the accompanying drawing, wherein is shown a preferred embodiment of the invention.

### BRIEF DESCRIPTION OF THE DRAWING

FIG. 1 is a flowchart describing the cellular regeneration process of treated cells.

FIG. 2 is flowchart describing the treatment process of damaged cells using the method of the present invention.

FIG. 3 is a diagram depicting a sample treatment area for an individual using the preferred method of treatment.

FIG. 4 is a diagram depicting multiple hand pieces being used to treat a patient in an alternative treatment method.

FIG. 5 is a diagram depicting another alternative treatment method in which two lasers are focused on overlapping treatment areas of a patient.

### DETAIL DESCRIPTIONS OF THE INVENTION

All illustrations of the drawings are for the purpose of describing selected versions of the present invention and are not intended to limit the scope of the present invention.

The present invention is a method for the treatment of living biological tissue by means of optical irradiation. The present invention is intended to treat soft tissue, however, similar methods may be adapted to treat hard tissue as well. The method for the treatment of living biological tissue utilizes a laser device to irradiate damaged cells. In the preferred embodiment of the present invention, the laser device is a hand piece that is made to be portable, however, it may also take the form of a fixed structure. A mounting structure can also be used such that the hand piece may be used from a fixed position.

The invention proposes multiple sequential surface area irradiations in a manner to expose the maximum number of underlying affected “reactive” cells. This sequential irradiation process should include major blood cell concentrations in vascular structures as well as specific organ sites. Special attention should be made to irradiation of all structures of the immune system, including but not restricted to the lymphatics, lymph nodes, spleen, skin, digestive tract, bone marrow, thymus.

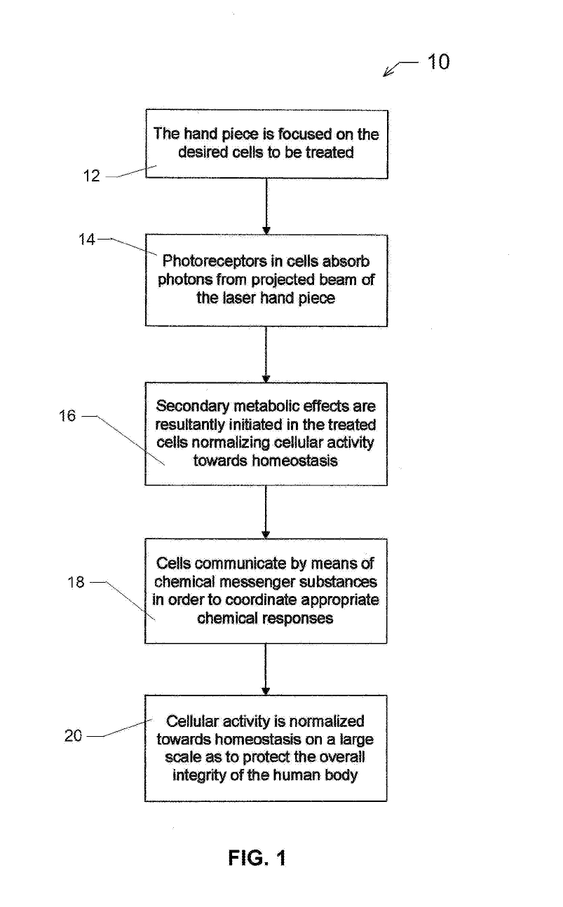

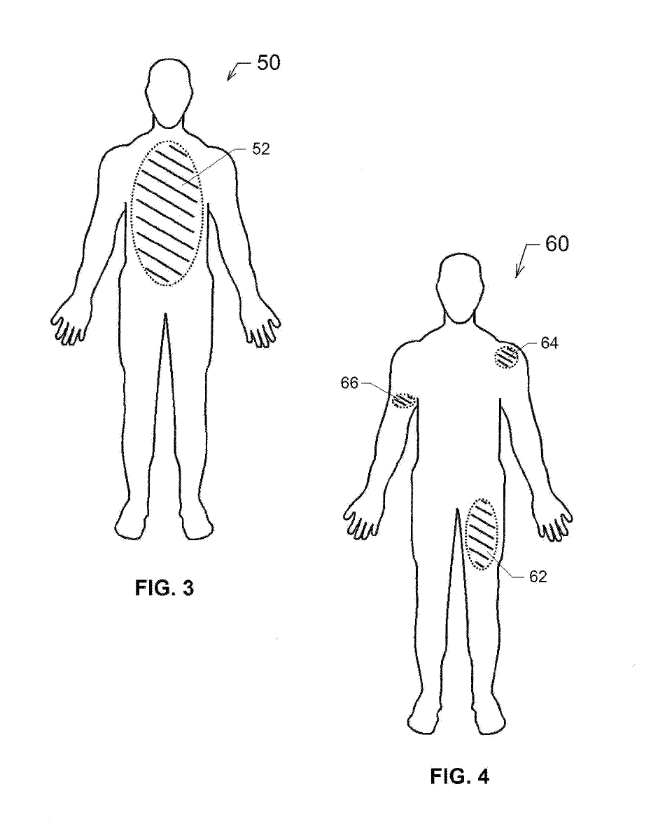

In reference to FIG. 1 and to FIG. 3, in the present invention, laser irradiation is utilized to alleviate the physical symptoms associated with allergic, auto-immune reactions, and immune deficiency conditions 10. Symptoms such as wheezing, urticaria, nervous system degeneration, joint and muscular skeletal changes, and specific organ involvement are results of over reactive or aberrant immune cell reactions. A hand piece is focused 12 on the cells 52 of a patient 50 that are to be treated. Photoactivation 14 of intracellular photoreceptors initiates a cascade 16 of secondary cellular metabolic effects, normalizing cellular activity towards homeostasis. This homeostasis is a fragile balance related to the reactive condition of adjacent cells. It is preferred to treat as many reactive cells as possible at or near the same time interval so as to generate a preponderance of neutral or homeostatic cell responses en masse. The amount of time and intensity of treatment is determined by the character of the cells to be treated, the depth of penetration desired, the chronicity of the condition, and the physical condition of the patient. Any number of factors in addition to those described above may be used to determine the operating levels of the hand piece such that it is operated below the photoablation threshold of the tissue.

The use of low level laser in the present invention acts to stimulate cellular regeneration, stabilize cell membranes, stabilize the indices of red blood cell deformation, increase lymphocyte counts, stimulate intracellular metabolism through mitochondrial photoreceptors, and stimulate 18 the production of intercellular messenger proteins and enzymes, specifically superoxide dismutase and catalase enzymes. Additionally, there is immediate increase in membrane permeability of nerve cells and regeneration of Schwann cells lining the nerves. RNA and subsequently DNA production is enhanced. Singlet 02 is also produced which further contributes to cellular regeneration.

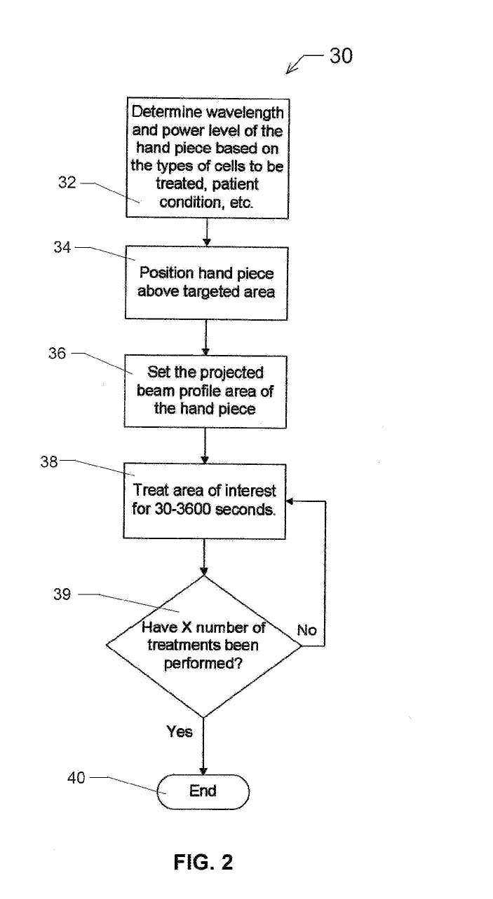

When these responses are exaggerated or erroneous as in the case of autoimmune disease and inflammation a violent cascade of cellular reactions contributes to biological changes which result in ongoing messenger signaling and elicits ongoing reactive cellular metabolic responses. The rapid communication 18 between immune and body cells clarifies the need for the preferred method to initiate, through photoactivation of cellular photoreceptors of large masses of cells a homeostatic intracellular metabolism and messaging. As intercellular messaging indicates homeostatic status 20, the reactive status of cells stabilizes through the various intracellular secondary metabolic effects and normalcy resumes. In reference to FIG. 2 and to FIG. 3, the hand piece is directed 34 at the damaged cells 52 of a patient 50 while configured to the appropriate wavelength and power level 32. The wavelength that the hand piece is operated at, as well as the duration of treatment 32 is dependent on the depth and type of cells being treated. The hand piece is ideally operated in the infrared spectrum between the wavelengths of 1060 nm to 1320 nm, however, the hand piece may be operated at any other wavelength. The hand piece can be operated at any level of power 32, with preference given to the 350 mW to 1200 mW range or a preferred range of from about 750 mW to about 2.8 Watts per square centimeter. The hand piece should be operated at a total power of between five watts and sixty watts, but may be operated at any total power level. In the preferred embodiment of the present invention the hand piece is operated at a fundamental wavelength of 1064 or 1275 nm and within the 750 mW to 1200 mW range or a preferred range of from about 750 mW to about 2.8 Watts per square centimeter. The hand held laser can be operated in a continuous or pulsed mode. In the preferred embodiment of the present invention the hand piece has a homogenous beam profile between one square centimeter and sixty square centimeters of surface area irradiation. The area of surface irradiation is crucial to the efficient radiation of a high volume of cells concurrently. As previously noted it is felt necessary to overcome cell numbers in a “reactive” state to benefit from the secondary benefits of photostimulation involving chemical messaging between cells. The projected beam may also be non-homogenous and may have a projected surface area less than or greater than the range described above. In the preferred embodiment of the present invention, the treatment duration range 38 for a single treatment session is between 30 seconds and 3600 seconds, however, it is also possible for the treatment duration to be shorter or longer. For each treatment, the hand piece should be configured such that it is operated below the photoablation threshold of the tissue being treated.

Photostimulation through the preferred method specifically activates the photoreceptors of cell membranes. This initiates ATP production in the mitochondria. The increased cellular energy in the form of ATP is then used by the cell to finance cellular metabolic needs as the cell moves metabolically towards homeostasis as is determined by genetic determination of cell type and function. The homeostatic cell continuously communicates with adjacent and even distant cells by sending and receiving chemical messenger substances. These messenger substances relate cell status to adjacent and distant cells and coordinate appropriate chemical responses to protect the integrity of the body overall.

Additional surface points of irradiation may also overlay cellular structures involved in cellular energy deficits secondary to or directly resulting from involvement in auto immune and immune mediated inflammatory reactions. These include but are not restricted to arterial endothelial cells, thyroid gland cells, pancreatic cells, liver cells, intestinal mucosal cells, brain cells and meninges, nerve cells, nerve ganglia cells, spinal cord cells, muscle cells, bone cells, cartilage cells, connective tissue cells, specialized respiratory cells, fat cells, and mucosal cells. These surface areas overlying the reactive cells may be irradiated concurrently or sequentially.

When immune system cells are actively engaged in creating antigen antibody reactions they release chemical messengers. These chemical messengers create the inflammatory cascade involving many other immune system cells that represents the classic immune system reaction to foreign substances. This cascade of the immune system response also involves local cell types as the inflammatory response engulfs an area. This involvement in a classical immune system reaction is in the form of energy depleting to the cells involve, while intracellular metabolism is shifted away from homeostasis toward messenger instruction mediated reactivity. Long term resolution of cascaded immune inflammatory reactions requires stopping the cascade inflammatory stimulation while addressing the energy deficit of cells already impacted by the immune messenger chemicals. Photostimulation of cells supplies energy for resumption of normal homeostatic cell metabolism which in turn involves the release of chemical messengers directing adjacent and distant cells toward biological equilibrium or homeostasis.

Specific immune system cellular reactions can be treated in situ at the point of immune mediated inflammation with appropriate time and dose related treatments. In addition the preferred method specifies the in situ irradiation of areas of high concentration of vascular structures containing mobile immune cells whose metabolic status may be of a reactive nature. Irradiation of vascular structures in a time related and dose specific fashion with a wavelength that is capable of penetrating to the depth of a large volume of vascular structures is best to irradiate the largest number of cells within those structures.

In reference to FIG. 4, as an alternative to the preferred method of treating a patient 60, multiple hand pieces may be used concurrently to irradiate acutely affected cells as well as cells within the bloodstream moving through key vascular areas 62, 64 and 66 of high blood cell concentration.

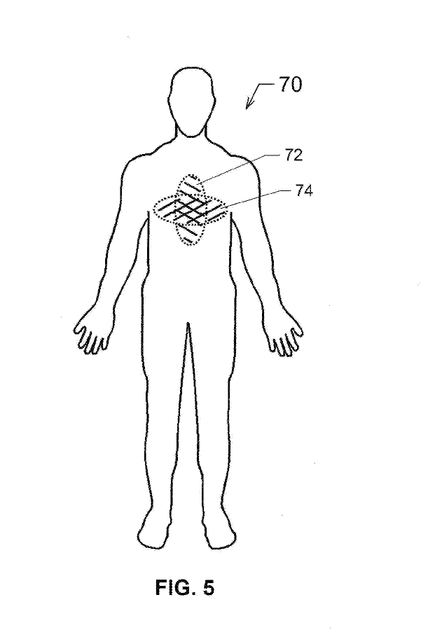

In reference to FIG. 5, as an alternative to the preferred method for treating a patient 70 two or more hand pieces may be used concurrently in relative close proximity 72 and 74 on the skin surface. Utilizing the deep penetration of the 1064 or 1275 nm wavelength and high power densities maintained below the level of cellular ablation, it is possible to increase the density of photon concentration to deep body cells by overlapping the projected conical distribution of laser light to coordinate with the depth of cells.

The invention advocates the use of a homogenous or non-homogenous beam of 1060 to 1325 nm laser light consistent with that of the laser. The invention claims benefits from the use of between 1060 nm and 1325 nm wavelengths of infrared light. The preferred method specifically uses 1064 or 1275 nm wavelengths.

The invention claims deep tissue penetration from 1060 to 1325 nm wavelength laser light in accordance with established models which illustrate preferable low absorption rates in melena, hemoglobin, and water at 1060 to 1325 nm wavelengths and more specifically at 1064 or 1275 nm wavelengths.

The invention claims that larger homogenous beam profile accompanied by higher power source results in larger total three dimensional area of cells to be irradiated. Cells are irradiated at powers less than that of cellular ablation.

The invention claims that only through high powers, wide beam profile and specific wavelengths can the largest number of cells be irradiated concurrently.

The invention claims that concurrent irradiation of large numbers of cells initiates photoactivation of preceptors in all cells irradiated.

The invention claims that concurrent activation of photoreceptors in cells, cell metabolism moves toward physiologic equilibrium concurrently.

The invention claims that secondary cell functions occur as a result of stimulation of cellular photoreceptors and the concurrent enhancement of cellular energy status as well as the resumption of more normal cellular metabolic activity is the basis for healing initiated by specifically using the 1064 or 1275 nm, 305 mw to 1200 mW range or a preferred range of from about 750 mW to about 2.8 Watts per square centimeter with a beam profile between 1 cm2 and 60 cm2.

Although the invention has been explained in relation to its preferred embodiment, it is to be understood that many other possible modifications and variations can be made without departing from the spirit and scope of the invention as hereinafter claimed.

1. A method of laser irradiation for alleviating the physical symptoms associated with allergic, auto-immune reactions, and immune deficiency conditions comprising the steps of:
focusing a hand piece on the cells that are to be treated;
photoactivating of intracellular photoreceptors by means of the hand piece, thereby initiating a cascade of secondary cellular metabolic effects; and
normalizing cellular activity towards homeostasis.

2. A method of laser irradiation for alleviating the physical symptoms associated with allergic, auto-immune reactions, and immune deficiency conditions according to claim 1 further including:
treating as many reactive cells as possible at or near the same time interval so as to generate a preponderance of neutral or homeostatic cell responses en masse.

3. A method of laser irradiation for alleviating the physical symptoms associated with allergic, auto-immune reactions, and immune deficiency conditions according to claim 2 further including:
stimulating the production of intercellular messenger proteins and enzymes, specifically superoxide dismutase and catalase enzymes.

4. A method of laser irradiation for alleviating the physical symptoms associated with allergic, auto-immune reactions, and immune deficiency conditions comprising the steps of:
directing a hand piece at the damaged cells;
configuring to the appropriate wavelength and duration for the hand piece, depending on the depth and type of cells to be treated; and
configuring to the appropriate power level for the hand piece.

5. A method of laser irradiation for alleviating the physical symptoms associated with allergic, auto-immune reactions, and immune deficiency conditions according to claim 4, wherein:
the step of configuring the appropriate wavelength for the hand piece configures in the infrared spectrum between the wavelengths from about 1060 nm to about 1320 nm.

6. A method of laser irradiation for alleviating the physical symptoms associated with allergic, auto-immune reactions, and immune deficiency conditions according to claim 5, wherein:
the step of configuring the appropriate power level for the hand piece, configures in one of the ranges per square centimeter of from about 750 mW to 1200 mW or from about 350 mW to about 1200 mW or from about 750 mW to about 2.8 Watts.

7. A method of laser irradiation for alleviating the physical symptoms associated with allergic, auto-immune reactions, and immune deficiency conditions comprising the steps of:
directing a hand piece at the damaged cells;
configuring to the appropriate fundamental wavelength of 1064 or 1275 nm and duration for the hand piece, depending on the depth and type of cells to be treated; and
configuring to the appropriate power level for the hand piece in one of the ranges of from about 750 mW to about 1200 mW or from about 750 mW to about 2.8 Watts.

8. A method of laser irradiation for alleviating the physical symptoms associated with allergic, auto-immune reactions, and immune deficiency conditions according to claim 7, further including the step of:
operating the hand held laser in a continuous mode and a homogenous beam profile in a range from about one square centimeter to about sixty square centimeters of surface area irradiation; and
treating for a single treatment session in a range from about 30 seconds to about 3600 seconds;
wherein the hand piece should be configured such that it is operated below the photoablation threshold of the tissue being treated.

9. A method of laser irradiation for alleviating the physical symptoms associated with allergic, auto-immune reactions, and immune deficiency conditions according to claim 7, further including the step of:
operating the hand held laser in a pulsed mode and a homogenous beam profile in a range from about one square centimeter to about sixty square centimeters of surface area irradiation; and
treating for a single treatment session in a range from about 30 seconds to about 3600 seconds;
wherein the hand piece should be configured such that it is operated below the photoablation threshold of the tissue being treated.

10. A method of laser irradiation for alleviating the physical symptoms associated with allergic, auto-immune reactions, and immune deficiency conditions, comprising the steps of:
directing a plurality of hand pieces at the damaged cells;
configuring to the appropriate wavelength and duration for each hand piece of the plurality of hand pieces, depending on the depth and type of cells to be treated;
configuring to the appropriate power level for each hand piece of the plurality of hand pieces; and
concurrently irradiating with the plurality of hand pieces.

11. A method of laser irradiation for alleviating the physical symptoms associated with allergic, auto-immune reactions, and immune deficiency conditions, according to claim 10, wherein:
the step of concurrently irradiating with the plurality of hand pieces comprises irradiating with the hand pieces in relative close proximity on the skin surface at 1064 or 1275 nm wavelength and high power densities maintained below the level of cellular ablation.
